(19) United States
(12) Patent Application Publication
(10) Pub. No.: US 2002/0131985 A1
(43) Pub. Date: Sep. 19, 2002

(54) System for customizing personal care products

(75) Inventors: May Shana'a, Fort Lee, NJ (US); Michael Charles Cheney, Fairfield, CT (US); Robert Avena, New York, NY (US); Kevin Keating, Darien, CT (US); Craig Stephen Slavtcheff, Guilford, CT (US); Brian John Dobkowski, Milford, CT (US); Rosa Paredes, Shelton, CT (US); Marion Louise Margosiak, Hamden, CT (US); Christy Bridges, Fairfield, CT (US); Marcina Siciliano, New Haven, CT (US)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 09/930,320

(22) Filed: Aug. 15, 2001

Related U.S. Application Data
(60) Provisional application No. 60/227,807, filed on Aug. 25, 2000.

(51) Int. Cl. A61K 7/00; G06F 17/60

(52) U.S. Cl. 424/401; 705/26

(57) ABSTRACT

A system is described for providing a customized, personal care product to a consumer at a location, typically the point of sale, that is remote from a second location in which a personal care product base composition is prepared. A selection of performance agents of different classes with a common vehicle is made available to the consumer for custom formulation of a personal care product with the desired attributes. The system typically includes a product base, a fragrance part, and one or more performance agent parts that may contain benefit agents, botanical extracts, active ingredients for skin and hair, and the like. A blank vehicle is substituted if insufficient performance agents are selected.

### PRIORITY

This application claims priority to provisional application No. 60/227,807 filed Au. 25, 2000.

### BACKGROUND OF THE INVENTION

Many personal care products such as body wash, facial cleansing gel, hair shampoo, hair conditioner, face lotion and other personal care products that are currently available are provided as prepared formulations. A drawback of such products is that the user cannot alter the formulation to accommodate their particular skin and hair characteristics, personal preferences, or to provide specialized treatment. A further drawback is the uncertainty of the age and freshness of the prepared formulation which may have been prepared many months or years before the product is sold.

Multiple-part product kits that contain separate components of e.g. a hair shampoo, hair conditioner, cosmetics, or other personal care products are available from many professional salons and some retail personal care outlets. However, the user is still unable to customize the individual formulations in light of the consumer's unique personal care preferences and requirements.

Therefore, one aspect of the invention is to provide a system that enables a user to formulate a variety of personal care product compositions to best suit the needs of the consumer. Another aspect of the invention is to provide a system that is easy to use such that the user may be a layperson who is able formulates a customized personal care product. Another aspect of the invention is to provide a plurality of performance agents in a common vehicle, which are compatible with the personal care product base, and form a stable personal care product, which is freshly blended.

### SUMMARY OF THE INVENTION

A system is provided for preparing a customized, personal care product for a consumer at a location remote from a second location in which a personal care product base composition is prepared, preferably at the point of retail sale. The system comprises providing a selection of personal care base compositions and a plurality of variants from two or more classes of performance agents whereby the variants of any one class have at least two ingredients in common with each other, and which are compatible with each other when all the components are mixed together. A plurality as used herein is defined as a number greater than one. Variants as used herein are defined as distinct members of a single class of performance agent which may be selected from such classes as botanical extracts, emollients, vegetable oils, active agents for treating or preventing skin disorders, vitamins, and the like. Variants may differ from each other with respect to either the identity (e.g. vitamin E acetate and vitamin A palmitate), or in the concentration of the aforementioned components.

Preferably the second class of performance agent has at least three ingredients in common. The consumer is allowed to select, in any sequence, one personal care base composition and at least two variants from separate classes of performance agents, such as a fragrance and a benefit agent. The user doses, in any sequence, the consumer selected personal care base composition, performance agents, and, if necessary, sufficient vehicle into a container, mixes the contents until uniform, and labels the container. The vehicle is flowable and may be a liquid, paste, flowable powder, or extrudable solid. Preferably the vehicle is a liquid with a viscosity ranging from water like to 100,000 cps at 25 C.

A sufficient quantity of a blank composition having at least two ingredients in common with at least one performance agent present in the product, and preferably having substantially the same vehicle composition as that of the performance agent may be dosed in the container if an insufficient number of performance agents are selected by the consumer. This allows for maintaining each of the following product characteristics: optimum concentration of base product ingredients, stability, viscosity, texture, clarity, and skin feel; while providing many choices for the consumer in customizing the product.

Inventive personal care product base compositions include cleansing and conditioning product bases useful for formulating body wash, body lotion, body mist spray, hydroalcoholic toner, facial cleansing gel, hand cleanser, hair shampoo, hair conditioner, a face lotion, a deodorant, a bar soap, a bath foam, bath salts, and the like.

### DETAILED DESCRIPTION OF THE INVENTION

In one aspect of the invention is a system for providing a customized, personal care product to a consumer at a location remote from a second location in which a personal care product base composition is prepared is provided. The system comprises the following:

(a) providing a selection from a plurality of said personal care base compositions in separate containers;

(b) providing a selection from a plurality of variants from a first class of performance agents in separate containers, each of said variants being delivered in a first vehicle, said first vehicle for each of said variants having at least two ingredients in common with each other, said first vehicle being compatible with a mixture of said personal care base composition and a second class of performance agents different from the first class;

(c) providing a selection from a plurality of variants of said second class of a performance agents in separate containers, each of said variants being delivered in a second vehicle, said second vehicle for each of said variants having at least two ingredients in common with each other;

(d) permitting the consumer to select, in any sequence, said at least one personal care base composition; at least one variant from said first class of performance agents; and at least one variant from said second class of performance agents;

(e) dosing, in a predetermined sequence, the consumer selected personal care base composition and performance agents into a container to form a personal care product; and

(f) mixing said personal care product until the product is uniform.

The predetermined sequence of dosing the personal care base and performance agents will normally be a random one i.e. where any sequence may be used. However, certain products will have greater stability if dosed in a specific sequence such as for body toner where the product base should be dosed first, for maximum stability.

In another aspect of the inventive method, a sufficient quantity of a blank composition is dosed into the product container, in substitution for at least one performance agent, whereby the final concentration of base product ingredients in the personal care composition is adjusted to be substantially equal to that of a final product where no substitution of said performance agents was made. Preferably the blank composition has at least two ingredients in common with any one of the performance agents contained in the final product.

Preferably the personal care product is provided to the consumer at the point of retail sale.

The personal care product base compositions useful in the invention typically contain one or more of the following: a solvent such as water, monohydric and/or polyhydric alcohols, polyethylene glycol, and the like; soaps, surfactants such as anionic, cationic, amphoteric, zwitterionc surfactants, and the like; conditioning agents such as cationic polymers, silicone polymers and the like; thickening agents such as acrylates, polysaccharide polymers, and the like; lathering aids such as alkanolamides and the like; emollients such as vegetable oils, fatty esters, and the like; pH adjusters, and preservatives. Further examples of useful solvents, surfactants, conditioning agents, thickening agents, lathering aids, emollients, pH adjusters, and preservatives are listed in the CTFA Cosmetic Ingredient Handbook, second edition, 1990; which is here incorporated by reference.

Other examples of useful anionic, amphoteric, and zwitterionic surfactants, and foam boosters of the personal care product base compositions useful in the invention are described in U.S. Pat. No. 5,221,530 issued to Janchitraponvej, et. al. on Jun. 22, 1993; incorporated herein by reference. Examples of useful hair shampoo, conditioner, and styling base compositions useful in the invention are described in U.S. Pat. No. 5,993,792 issued to Rath, et. al. on Nov. 30, 1999 incorporated herein by reference. Preferably the personal care product base has a viscosity in the range of about 0.9 to 100,000 cps at 25 C, more preferably in the range of about 0.9 to 30,000 cps at 25 C.

The viscosity of the product base can be varied, from a pourable liquid, to a thick paste or extrudable material, depending on its composition and the amount of thickener added to the base. The product base may also comprise a flowable powder.

In another aspect of the invention, a customized label is applied to the product container which identifies the product and only the components of the base formula and dosed performance agents actually contained in the container. Such a label may be generated by any suitable means, but is preferably generated by a computer interfaced with a printer which allows for the user to input the consumer's custom product selections and accesses a database of ingredients contained in the available product bases and performance agent compositions to produce a customized label. The same computer printer combination may also be used to insert a code on the label, e.g. a machine readable bar code or the like, capable of tracking the identity of both the product and the consumer for later reference. In this way, the consumer can easily reorder the custom product from the same location the product was purchased or from a remote location. The consumer may also return to the store or kiosk and allow the previous label to be scanned thus facilitating the purchase of additional product.

One class of performance agents may typically be fragrances, and may contain a solvent, such as water, a monohydric alcohol, a polyhydric alcohol or a blend thereof. Among suitable monohydric alcohols are ethanol, isopropanol, butanol, hexanol and combinations thereof, and the like. Most preferred is ethanol. Suitable polyhydric alcohols include glycerin (known also as glycerol), propylene glycol, dipropylene glycol, polyethylene glycol, sorbitol, hydroxypropyl sorbitol, hexylene glycol, 1,3-butylene glycol, isopropylene glycol and mixtures thereof, and the like. Most preferred are propylene glycol and polyethylene glycol.

This class of performance agent also contains at least one preservative, preferably one or more of a biocide, a chelate, an UV inhibitor and an antioxidant or any combination thereof. More preferably the fragrance agent contains DMDM Hydantoin, Iodopropynyl Butylcarbamate, and EDTA, and an UV inhibitor. Another aspect of the invention is the availability of fragrance and other performance agent intensity levels that are selectable by the consumer. Further examples of useful preservatives, chelates, antioxidants, and UV inhibitors are listed in the CTFA Cosmetic Ingredient Handbook, second edition, 1990.

Other classes of performance agents of the invention may include a colorant, and a benefit agent. Inventive benefit agents include emollients, botanical extracts, vitamins, other active ingredients to prevent or treat undesired skin and hair conditions, and the like. Additionally, the inventive benefit agents contain a solvent such as water, a monohydric alcohol, or a polyhydric alcohol, e.g. propylene glycol or the like; a solubilizing agent, and at least one preservative. Preferably the solubilizing agent is selected from at least one of a polyethylene glycol ether of a fatty alcohol, a polyethylene glycol ether of hydrogenated castor oil, a polyethylene glycol derivative of a sorbitan ester, a polysorbate, a glycerol ester, a polyethylene glycol derivative of a glycerol ester, an alkyl phosphate, and an alkyl sulfate, and the like. Further examples of useful benefit and solubilizing agents are listed in the CTFA Cosmetic Ingredient Handbook, second edition, 1990. Preferably preservatives consist of one or more of a biocide, a chelate, an UV inhibitor, and an antioxidant or any combination thereof. More preferably, the performance agent preservatives are selected from DMDM Hydantoin, Iodopropynyl Butylcarbamate, polyaminocarboxylic acid chelates or their salts such as EDTA, and the like. The inventive blank performance agent, or vehicle, contains substantially no fragrance or benefit agent, and has at least two ingredients in common with at least one of the other performance agents in its class, including preferably a solvent, and one or more solubilizing agents. Preferably the blank benefit agent has at least three ingredients in common with the other benefit agents including preferably a solvent, one or more solubilizing agents, and a preservative.

In another aspect, the inventive system produces custom products of convenient volume to facilitate consumer use. Preferably, the container for the final product has a volume under about 1 liter. Preferably the container has a neck to facilitate pouring and is threaded or flanged to accept a cap, pump, or the like. The container may be opaque, translucent, or transparent; in whole or in part, and made of any suitable material which is not attacked by the contents of the product contained therein. Suitable materials include glass, plastic, barrier-coated paper, metal, or composites or laminates thereof. Preferably the product container is made of polyethylene, polypropylene, or PET; and is transparent or translucent. Prior to mixing the contents of the container, preferably either a cap without an integral orifice is used to cap the container or a plug is inserted in the container's neck after the container has been filled to a level below the neck using a plug that's sized to occupy at least 50% of the neck volume. This is done in part to improve mixing efficiency when the container's contents are blended by e.g. an external mixing device, such as a Red Devil model 5300 twin axis mixer (Brooklyn Park, Minn.) or the like. Preferably the container is situated in the mixer in a position at an angle of greater than 10 degrees, more preferably greater than 30 degrees, from its major axis vertical orientation, and most preferably in a position substantially inverted from its filling position.

Except in the operating and comparative examples, or where otherwise explicitly indicated, all numbers in this description indicating amounts of material ought to be understood as modified by the word “about”.

The following examples will more fully illustrate the embodiments of this invention. All parts, percentages and proportions referred to herein and in the appended claims are by weight unless otherwise illustrated.

### EXAMPLE 1

The custom formulation of separate embodiments of an inventive body lotion, hair conditioner, facial cleanser, body mist, toner, body wash, instant hand cleanser, hair shampoo and face lotion are illustrated in Table 1A. Specifically illustrated is the choice of a single base formula corresponding to the product type above, the choice of one of three different embodiments of inventive fragrance concentrates, or alternatively, of a single, inventive fragrance concentrate, to be dispensed either in one, two or three portions in order to provide moderate, strong, and stronger fragrance intensity levels, and the choice of one or more of four different embodiments of inventive benefit agents. Tables 1B and 1C illustrate embodiments of the inventive fragrance vehicle or blank and the inventive benefit vehicle or blank respectively.

### EXAMPLE 2

Table 2 shows in further detail the composition of the embodiment of the custom body lotion product base depicted in table 1. Table 2a shows the composition of a body lotion base concentrate, tables 2b and 2c show the composition of embodiments of an inventive fragrance and benefit agent concentrate respectively that the consumer may select, and table 2d shows how the consumer selected agents are blended together to make the custom body lotion product. If the-consumer does not pick sufficient choices of a fragrance, benefit agent, or other performance agent; sufficient inventive vehicle or blank fragrance/benefit agents, as depicted in tables 2e and 2f respectively, are substituted.

### EXAMPLE 3

Table 3a shows in further detail the composition of an embodiment of the custom hair conditioner product base depicted in table 1. Inventive fragrance and benefit agents are depicted in tables 2b and 2c above. Table 3b shows how the consumer selected agents are blended together to make the custom hair conditioner product. As for the body lotion in Example 2, if the consumer does not pick sufficient choices for a fragrance or benefit agent for the hair conditioner, sufficient inventive vehicle or blank fragrance/benefit agents as depicted in tables 2e and 2f respectively are substituted.

TABLE 1A

| | | Fragrance | Fragrance | Fragrance | Benefit #1 | Benefit #2 | Benefit #3 | Benefit #4 |
|---|---|---|---|---|---|---|---|---|
| | | Allotment #1 | Allotment #2 | Allotment #3 | Solvent (Water) | Solvent (Water) | Solvent (Water) | Solvent (Water) |
| | | Solvent (Propy- | Solvent (Propy- | Solvent (Propy- | Solubilizing | Solubilizing | Solubilizing | Solubilizing |
| | | lene Glycol) | lene Glycol) | lene Glycol) | Agent (PEG-40 | Agent (PEG-40 | Agent (PEG-40 | Agent (PEG-40 |
| | | Preservatives | Preservatives | Preservatives | Hydrogenated | Hydrogenated | Hydrogenated | Hydrogenated |
| | | (Glydant Plus | (Glydant Plus | (Glydant Plus | Castor Oil & | Castor Oil & | Castor Oil & | Castor Oil & |
| | | & Edta) | & Edta) | & Edta) | Trideceth-9) | Trideceth-9) | Trideceth-9) | Trideceth-9) |
| | | UV-Inhibitor | UV-Inhibitor | UV-Inhibitor | Preservatives | Preservatives | Preservatives | Preservatives |
| | | (Benzo- | (Benzo- | (Benzo- | (Glydant Plus | (Glydant Plus | (Glydant Plus | (Glydant Plus |
| | | phenone-2) | phenone-2) | phenone-2) | & Edta) | & Edta) | & Edta) | & Edta) |
| Product | Base | Color & | Color & | Color & | Performance | Performance | Performance | Performance |
| Form | Formula | Fragrance | Fragrance | Fragrance | Agent (Benefit) | Agent (Benefit) | Agent (Benefit) | Agent (Benefit) |
| Body | 90.435% | .87% | .87% | .87% | 1.74% | 1.74% | 1.74% | 1.74% |
| Lotion | | | | | | | | |
| Hair | 91.478% | .522% | .522% | .522% | 1.74% | 1.74% | 1.74% | 1.74% |
| Con- | | | | | | | | |
| ditioner | | | | | | | | |
| Facial | 92.2636% | .26% | .26% | .26% | 1.74% | 1.74% | 1.74% | 1.74% |
| Cleanser | | | | | | | | |
| Body | 86% | 2% | 2% | 2% | 2% | 2% | 2% | 2% |
| Mist | | | | | | | | |
| Toner | 91.1% | .3% | .3% | .3% | 2% | 2% | 2% | 2% |
| Body | 89.5% | 1.33% | 1.33% | 1.33% | 1.625% | 1.625% | 1.625% | 1.625% |
| Wash | | | | | | | | |
| Instant | 83.49% | .56% | .56% | .56% | 3.71% | 3.71% | 3.71% | 3.71% |
| Hand | | | | | | | | |
| Cleanser | | | | | | | | |
| Hair | 90.8% | .84% | .84% | .84% | 1.675% | 1.675% | 1.675% | 1.675% |
| Shampoo | | | | | | | | |
| Face | 93.03% | .084% | .084% | .084% | 1.68% | 1.68% | 1.68% | 1.68% |
| Lotion | | | | | | | | |

TABLE 1B

Fragrance Blank
Solvents (Dipropylene Glycol & Propylene
Glycol)
Preservatives (Glydant Plus &
Tetrasodium Edta)
UV-Inhibitor (Benzophenone-2)
NO COLOR

TABLE 10

Benefit Blank
Solvent (Water)
Solubilizing Agent
(PEG -40 hydrogenated Castor Oil and
Trideceith-9)
Preservatives (Glydant Plus and
Disodium EDTA

### Tables 2 a-f

TABLE 2a
Base Hand Lotion formula

| COMPONENT | % wt/wt |
|---|---|
| WATER | 75-80% |
| Na2 EDTA | .04-.08 |
| magnesium aluminum silicate | .02-.04 |
| tio2 | .08-.12 |
| carbopol | 8-12 |
| Triethanolamine | 1-1.5 |
| Stearic Acid | 2-3 |
| Cetyl Alcohol | 2-3 |
| Mineral Oil | 2-3 |
| PEG-100 Stearate | .2-1.0 |
| steramide | .2-1.0 |
| Glyceryl Stearate | .2-1.0 |
| silicone | .2-1.0 |
| C12-15 Alkyl Ethylhexanoate | .2-1.0 |
| Glydant Plus | .1-5 |

TABLE 2d
Finished Hand Lotion Formula

| Ingredient | % wt/wt |
|---|---|
| Base Lotion Formula | 90.4 % |
| Fragrance Formula | 2.6 % |
| Benefits Formulas | 7.0 % |

TABLE 2b
Custom fragrance formula

| Ingredient | Function | % wt/wt |
|---|---|---|
| Fragrance | Performance | 40-60 % |
| | Agent | |
| Propylene Glycol | Solvent | 40-60 % |
| Glydant Plus liquid | Preservative | 0.1-0.5 % |
| Tetrasodium EDTA | Preservative | 0.01-0.2 % |
| Benzophenone-2 | UV-Inhibitor | 0.1-0.5 % |
| OPTIONAL COLORS | Performance | 0.01-3.0 % |
| | Agent | |

TABLE 2f
Custom fragrance blank formula

| Ingredient | Function | % wt/wt |
|---|---|---|
| Dipropylene Glycol | Solvent | 40-60 % |
| Propylene Glycol | Solvent | 40-60 % |
| Glydant Plus liquid | Preservative | 0.1-0.5 % |
| Tetrasodium EDTA | Preservative | 0.01-0.2 % |
| Benzophenone-2 | UV-Inhibitor | 0.1-0.5 % |

TABLE 2c
Custom benefit formula

| | | |
|---|---|---|
| Ingredient | Function | % wt/wt |

| | | |
|---|---|---|
| DI Water | Solvent | 93-97 |
| Benefit Component | Performance | 0.3-1.0 % |
| | Agent | |
| Solubilizer | Solubilizing Agent | 2-6 % |
| Glydant Plus liquid | Preservative | 0.1-0.5 % |
| Disodium EDTA | Preservative | 0.01-0.1 % |

TABLE 2e
Custom benefit blank formula

| Ingredient | Function | % wt/wt |
|---|---|---|
| DI Water | Solvent | 93 - 97 |
| Solubilizer | Solubilizing Agent | 2 - 6 % |
| Glydant Plus liquid | Preservative | 0.1 - 0.5 % |
| Disodium EDTA | Preservative | 0.01 - 0.1 % |

TABLE 3a
Base Hair Conditioner formulation

| Ingredients | % wt/wt |
|---|---|
| DI Water | 80-90 |
| Citric Acid | 0.1-0.4 |
| Hydroxyethylcellulose | 0.1-0.4 |
| Stearamidopropyl Dimethylamine | 0.2-1.0 |
| Stearyl Octyldimonium Methosulfate | 1.0-2.0 |
| BES & Stearyl Alcohol | 0.2-1.0 |
| Cetyl Alcohol | 1.5-4.0 |
| Stearyl Alcohol | 1.0-2.0 |
| Disodium EDTA | 0.1-0.4 |
| Potassium Hydroxide | 0.01-0.1 |
| Glydant Plus Liquid | 0.1-0.4 |
| dimethyl siloxane derivative & | 0.7-2.0 |
| TEA-Dodecylbenzenesulfonate | |
| Siloxane derivative | 0.7-2.0 |

TABLE 3b
Finished Hair Conditioner Product

| Ingredient | % wt/wt |
|---|---|
| Base Conditioner | 91.5 % |
| Formula | |
| Fragrance Formula | 1.5 % |
| Benefits Formulas | 7.0 % |

The foregoing description and examples illustrate selected embodiments of the present invention. In light thereof variations and modifications will be suggested to one skilled in the art, all of which are within the scope and spirit of this invention.

We claim:

1. A system for providing a customized, personal care product to a consumer at a location remote from a second location in which a personal care product base composition is prepared, comprising:
(a) providing a selection from a plurality of said personal care base compositions;
(b) providing a selection from a plurality of variants from a first class of performance agents, each of said variants being delivered in a first vehicle, said first vehicle for each of said variants having at least two ingredients in common with each other, said first vehicle being compatible with a mixture of said personal care base composition and a second class of performance agents different from the first class;
(c) providing a selection from a plurality of variants of said second class of a performance agents, each of said variants being delivered in a second vehicle, said second vehicle for each of said variants having at least two ingredients in common with each other;
(d) permitting the consumer to select, in any sequence, said at least one personal care base composition; at least one variant from said first class of performance agents; and at least one variant from said second class of performance agents;
(e) dosing, in a predetermined sequence, the consumer selected personal care base composition and performance agents into a container to form a personal care product; and
(f) mixing said personal care product until the product is uniform.

2. The method of claim 1 wherein said second vehicle for each variant has at least three ingredients in common.

3. The method of claim 1 wherein a sufficient quantity of a blank composition is dosed in said container, in substitution for at least one performance agent, whereby the final concentration of base product ingredients in said personal care composition is adjusted to be substantially equal to that of a final product where no substitution of said performance agents was made.

4. The method of claim 3 wherein said blank composition has at least two ingredients in common with any one of said performance agents.

5. The method of claim 1 wherein said customized personal care product is selected from a body wash, a body lotion a body mist spray, a hydroalcoholic toner, a facial cleansing gel, a hand cleanser, a hair shampoo, a hair conditioner, a face lotion, a deodorant, a bar soap, a bath foam, and bath salts.

6. The method of claim 1 wherein a customized label is applied to the container identifying the product and only the components of the base formula and dosed performance agents contained therein.

7. The method of claim 1 wherein said first class of performance agents are fragrances.

8. The method of claim 7 wherein each of said fragrances contain a solvent and at least one preservative in common with each other.

9. The method of claim 8 wherein said solvent is selected from water, a monohydric alcohol, a polyhydric alcohol, or a blend thereof.

10. The method of claim 8 wherein said preservatives are selected from DMDM Hydantoin, Iodopropynyl Butylcarbamate, polyaminocarboxylic acid chelates or salts thereof, and phosphonate chelates.

11. The method of claim 7 wherein at least one fragrance selection has a plurality of scent intensity levels selectable by the consumer.

12.The method of claim 1 wherein said performance agents are selected from a fragrance, a colorant, and a benefit agent.

13. The method of claim 1 wherein said second class of performance agents are benefit agents.

14. The method of claim 13 wherein at least one benefit selection has a plurality of benefit intensity levels selectable by the consumer.

15. The method of claim 2 wherein said second vehicle's common ingredients include a solvent, a solubilizing agent, and a preservative.

16. The method of claim 14 wherein said solvent is selected from water, a monohydric alcohol, a polyhydric alcohol, or a blend thereof.

17. The method of claim 14 wherein said solubilizing agent is selected from at least one of a polyethylene glycol ether of a fatty alcohol, a polyethylene glycol ether of hydrogenated castor oil, a polyethylene glycol derivative of a sorbitan ester, propylene glycol, a polysorbate, a glycerol ester, a polyethylene glycol derivative of a glycerol ester, an alkyl phosphate and an alkyl sulfate.

18. The method of claim 14 wherein said preservatives are selected from DMDM Hydantoin, Iodopropynyl Butylcarbamate, polyaminocarboxylic chelates, and phosphonate chelates.

19. The method of claim 1, comprising dosing a third class of performance agent different from said first and second class of performance agent, said third class of performance agent having at least two ingredients in common with at least one of said first and second class of performance agents; said third class of performance agent being compatible with said product base, and said first and second class of performance agents.

20. The method of claim 6 wherein the label contains a code capable of tracking the identity of both the product and the consumer for later reference.

21. The method of claim 20 wherein the code is in the form of a machine scannable bar code.

22. The method of claim 1 where said container has a volume under about 1 liter.

23. The method of claim 22 wherein said container has a neck; a plug is inserted in said container's neck after said container has been filled to a level below said neck; said plugged neck is then capped; said plug occupying at least 50% of the volume of said neck to improve mixing efficiency when said container's contents are blended by a mixing device while said container is situated in a position substantially inverted from its filling position.

24. The method of claim 22 wherein said container has a neck; a cap without an orifice is used to cap said container to improve mixing efficiency when said container's contents are blended by a mixing device while said container is situated in a position substantially inverted from its filling position.

25. The method of claim 1 wherein said container is agitated while its major axis is positioned at an angle greater than 10 degrees from the vertical.

26. The method of claim 1 wherein said angle is greater than 30 degrees from the vertical.

27. The method of claim 1 wherein said personal care product base has a viscosity in the range of about 0.9 to 100,000 cps at 25 C.

28.The method of claim 1 wherein said product base's viscosity is in the range of about 0.9 to 30,000 cps at 25 C.

29.The method of claim 1 wherein said second location is a retail location.
